(19) United States
(12) Patent Application Publication
(10) Pub. No.: US 2026/0115440 A1
(43) Pub. Date: Apr. 30, 2026

(54) HANDPIECE FOR SKIN TREATMENT

(71) Applicant: EUNSUNG GLOBAL CORP., Wonju-si (KR)

(72) Inventor: Ki Se LEE, Seoul (KR)

(21) Appl. No.: 19/323,195

(22) Filed: Sep. 9, 2025

(30) Foreign Application Priority Data
Oct. 25, 2024 (KR) 10-2024-0147286

(51) Int. Cl. A61M 37/00; A61N 1/32

(52) U.S. Cl. CPC A61M 37/0015; A61N 1/327; A61M 2037/0007; A61M 2037/0023

(57) ABSTRACT

Proposed is a handpiece for skin treatment, which includes a main body housing, a needle cartridge, a needle driving part; a needle insertion depth adjustment part, and a fastening member, wherein the needle insertion depth adjustment part includes a first driving motor seated in the main body housing, a gear disposed on a shaft of the first driving motor, and a fastening nut seated in the main body housing, engaged with the gear, and configured to move rectilinearly forward toward the needle cartridge and backward therefrom according to an operation of the first driving motor.

### CROSS REFERENCE TO RELATED APPLICATION

The present application claims priority to Korean Patent Application No. 10-2024-0147286, filed Oct. 25, 2024, the entire contents of which is incorporated herein for all purposes by this reference.

### BACKGROUND

### Field of the Invention

The present disclosure relates to a handpiece for skin treatment which is used to address skin problems in dermatological, aesthetic, or plastic surgery clinics.

### Description of the Related Art

To maintain and improve the elasticity of human skin and to counteract skin aging, a drug injection method using the principle of electroporation may be used as a method that induces biological changes through efficacious substances.

The drug injection method using the principle of electroporation is mainly used for skin beauty and plastic surgery, and for the treatment of specific parts of the body. The drug injection method is a method to enable the penetration of substances with a certain molecular weight (less than 4000 Dalton) through the cell membrane. An electrical pulse disrupts the phospholipid bilayer, temporarily creating a hole on the skin surface, so that a drug enters the cell through the hole.

For drug administration through the skin, a microneedling handpiece may be used among various skin treatment handpieces.

A microneedling handpiece is used as a medical device that uses microneedles (fine needles) to promote skin regeneration and collagen production. The microneedling handpiece may be used as a medical device that helps with effective skin regeneration by combining high-frequency energy with microneedles to deliver the energy deep into the skin.

The above information may be provided as related art for the purpose of assisting in understanding the present disclosure. However, no claim or determination is made as to whether any of the above is applicable as prior art with respect to the present disclosure.

### Document of Related Art

(Patent Document 1) Korean Patent No. 10-0420731

### SUMMARY OF THE INVENTION

However, the conventional method of applying an electric pulse to the skin by bringing the electrode plate into contact with the skin to allow required drug to penetrate into cells has a low rate of the efficacy of the drug reaching the epidermis and dermis through the stratum corneum, and even if a formulation or chemical skin absorption enhancer is used to improve skin permeability, it may not produce satisfactory results if the drug is hydrophilic or has a molecular weight greater than 300 Daltons.

In addition, it may be difficult to control the depth of a needle penetrating human skin, making it difficult to actively respond to environments with various skin conditions.

In addition, conventional microneedling handpieces exhibit significant vibration-induced shaking during operation, which may cause discomfort during skin treatment.

In addition, conventional microneedling handpieces have a separate waterproof structure, resulting in vulnerability to moisture.

The technical problems to be solved by the present document are not limited to those mentioned above, and other technical problems not mentioned will be clearly understood by those skilled in the art from the following description.

As a technical means for solving the above-described technical problems, one aspect of the present disclosure is to provide a handpiece for skin treatment with improved drug absorption efficiency.

In addition, another aspect of the present disclosure is to provide a handpiece for skin treatment capable of adjusting the depth of needle penetration into the skin.

In addition, another aspect of the present disclosure is to provide a handpiece for skin treatment in which a shaft is assembled to a driven cam and the shaft and the driven cam are coaxially arranged by a bushing-shaped support member, thereby minimizing shaking during the operation of a driving motor.

In addition, another aspect of the present disclosure is to provide a handpiece for skin treatment that includes a waterproof structure, thereby counteracting moisture penetration into the interior of the device.

According to various embodiments of the present disclosure, there is provided a handpiece for skin treatment, the handpiece including: a main body housing; a needle cartridge including the needle tip accommodated by a needle housing and having a needle PCB, on which a plurality of needles are arranged in a protruding manner, with the needle cartridge replaceably disposed on a first end of the main body housing; a needle driving part coaxially connected to the needle cartridge within the main body housing and configured to provide a vibration force to the needles; a needle insertion depth adjustment part fastened coaxially to the needle driving part within the main body housing and configured to move the needle cartridge forward or backward; and at least one fastening member integrally connecting the needle driving part and the needle insertion depth adjustment part to each other, wherein the needle insertion depth adjustment part may include: a first driving motor seated in the main body housing; a gear disposed on a shaft of the first driving motor; and a fastening nut seated in the main body housing, engaged with the gear, and configured to move rectilinearly forward toward the needle cartridge and backward therefrom according to an operation of the first driving motor.

In the handpiece for skin treatment according to an embodiment, electrically energized needles repeatedly puncture the skin of a subject, thereby enhancing the penetration of a therapeutic drug applied to the skin, and enabling effective skin treatment through the wound-healing response of the dermis without damaging the epidermis.

In the handpiece for skin treatment according to an embodiment, the safety and accuracy of the procedure can be improved by simple adjustment of the penetration speed and insertion depth of a needle.

In the handpiece for skin treatment according to an embodiment, a waterproof function can be improved by the waterproof structure inside the housing.

In the handpiece for skin treatment according to an embodiment, the shaft is assembled to the driven cam, and the shaft and the driven cam are arranged concentrically by a bushing-shaped support member, thereby minimizing shaking during the operation of the driving motor.

The effects of the present disclosure are naturally exhibited by the described configuration, regardless of whether the inventor is aware of them. Accordingly, the aforementioned effects are merely some of the effects based on the disclosed content, and should not be construed as encompassing all effects identified by the inventor or that actually exist.

The effects obtainable from the present disclosure are not limited to those mentioned above, and other effects not explicitly stated will be clearly understood by those skilled in the art from the description below.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1A is a perspective view showing a handpiece for skin treatment according to an embodiment;

FIG. 1B is a partial cutaway cross-sectional view showing the handpiece for skin treatment according to an embodiment;

FIG. 2 is an exploded perspective view showing the configuration of a needle insertion depth adjustment part of the handpiece for skin treatment according to an embodiment;

FIG. 3 is a cross-sectional view showing the configuration of the needle insertion depth adjustment part of the handpiece for skin treatment according to an embodiment;

FIG. 4 is a plan view showing the assembled state of the needle insertion depth adjustment part of the handpiece for skin treatment according to an embodiment;

FIG. 5 is an exploded perspective view showing the configuration of a needle driving part of the handpiece for skin treatment according to an embodiment;

FIG. 6 is a plan view showing the assembled configuration of the needle driving part of the handpiece for skin treatment according to an embodiment;

FIG. 7 is a perspective view showing the assembled state of the needle driving part of the handpiece for skin treatment according to an embodiment;

FIG. 8 is an exploded perspective view showing the configuration of a needle cartridge of the handpiece for skin treatment according to an embodiment;

FIG. 9A is a front view showing the configuration of the needle cartridge of the handpiece for skin treatment according to an embodiment;

FIG. 9B is a side view showing the configuration of the needle cartridge of the handpiece for skin treatment according to an embodiment;

FIG. 9C is a rear view showing the configuration of the needle cartridge of the handpiece for skin treatment according to an embodiment; and

FIG. 10 is a front cross-sectional view showing the configuration of the needle cartridge of the handpiece for skin treatment according to an embodiment.

With regard to the description of the drawings, identical or similar reference numerals may be used for identical or similar components.

### DETAILED DESCRIPTION OF THE INVENTION

Hereinafter, embodiments will be described in detail with reference to the accompanying drawings so that those skilled in the art can easily implement the present disclosure. However, the disclosed embodiments may be implemented in various different forms and are not limited to the embodiments described herein.

Hereinafter, the configuration of a microneedle-type handpiece will be described with reference to the accompanying drawings.

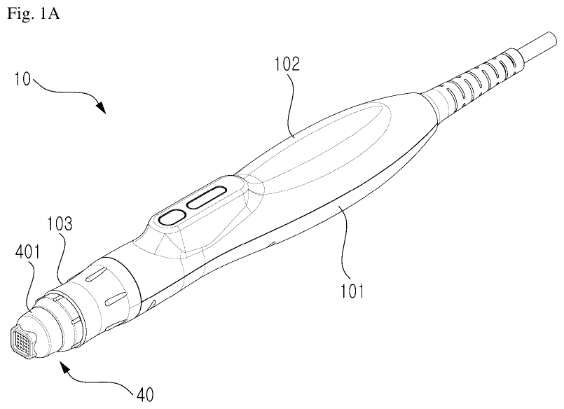

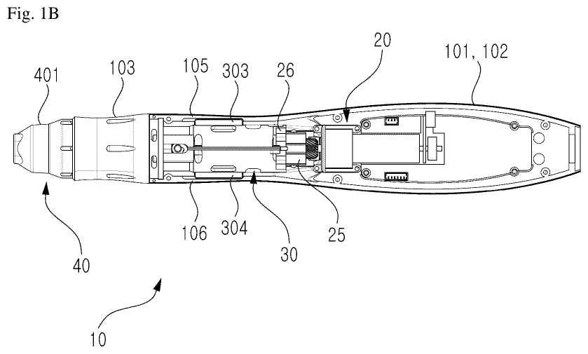

FIG. 1A is a perspective view showing a handpiece for skin treatment according to an embodiment. FIG. 1B is a partial cutaway cross-sectional view showing the handpiece for skin treatment according to an embodiment.

Referring to FIGS. 1A and 1B, human skin tissue is composed of the stratum corneum layer, the epidermis layer, the dermis layer, and the hypodermis layer. The function of the skin may deteriorate due to aging and exposure to ultraviolet radiation. Representative changes in the skin caused by aging and UV exposure include the formation of wrinkles due to the reduction and deformation of collagen fibers in the dermis, decreased skin elasticity due to the degeneration of elastic fibers composed of elastin, and melanin pigmentation resulting from the activation of tyrosinase present in the epidermis.

To maintain and improve skin elasticity and counteract aging, a drug injection method using the principle of electroporation, which is known as electric perforation, may be used as a method that can induce biological changes through efficacious substances.

The handpiece for skin treatment using the aforementioned drug administration method may be used as a medical device to address skin problems in dermatological, aesthetic, or plastic surgery clinics.

For example, skin problems in humans may include reduced skin elasticity, the formation of wrinkles, or acne scars.

Among the various handpieces for skin treatment listed above, the microneedling handpiece may be used as a medical device that employs microneedles (fine needles) to promote skin regeneration and collagen production. This type of microneedling handpiece piece has been used as a medical device that helps with effective skin regeneration by combining high-frequency energy with microneedles to deliver the energy deep into the skin.

According to an embodiment, a handpiece 10, which is a medical device for addressing skin problems in dermatological, aesthetic, or plastic surgery clinics, may include a replaceable bar-type needle cartridge 40 with an ergonomic grip, and a main body housing 100. According to an embodiment, the main body housing 100 may include first to third housings 101, 102, and 103. According to an embodiment, a needle insertion depth adjustment part 20 and a needle driving part 30 may be accommodated within the first and second housings 101 and 102, and at least a portion of components constituting the needle cartridge 40 may be accommodated within a needle housing 401 and the third housing 103.

According to an embodiment, in the handpiece 10, the needle insertion depth adjustment part 20, the needle driving part 30, and the needle cartridge 40 are sequentially arranged coaxially along a rotational axis (e.g., a driving motor rotational axis). According to an embodiment, the needle insertion depth adjustment part 20 and the needle driving part 30 may be coaxially coupled by using at least one fastening nut 25 and at least one fastening member 26, and the needle driving part 30 may be coaxially connected to the needle cartridge 40.

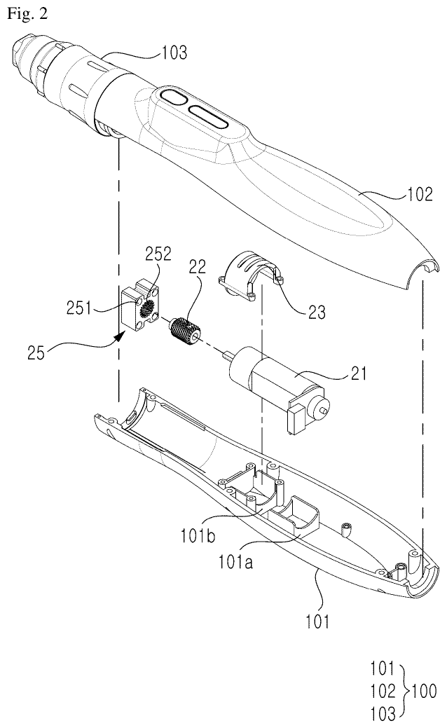

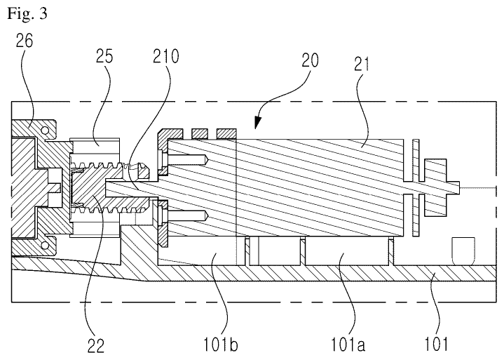

FIG. 2 is an exploded perspective view showing the configuration of a needle insertion depth adjustment part of the handpiece for skin treatment according to an embodiment. FIG. 3 is a cross-sectional view showing the configuration of the needle insertion depth adjustment part of the handpiece for skin treatment according to an embodiment.

Referring to FIGS. 2 and 3, the main body housing 100 according to an embodiment may include the first housing 101 and the second housing 102 fastened to the first housing 101. According to an embodiment, the main body housing 100 may accommodate the needle driving part 30 and a needle insertion depth adjustment part 20.

According to an embodiment, the needle insertion depth adjustment part 20, which is a driving device that moves the needle driving part 30 toward or away from the needle cartridge 40, may be integrally fixed to the needle driving part 30 by a fastening structure and may be seated within the main body housing 100. According to an embodiment, the fastening structure may serve as a fixing device that integrally connects one end of the needle driving part 30 to one end of the needle insertion depth adjustment part 20, and may include the fastening nut 25 and a fastener (not shown). According to an embodiment, the fastening nut 25 may include four fastening holes 251 formed at four corner portions extending in the forward or backward direction of the needle, and a gear hole 252 into which a gear 22 included in the needle insertion depth adjustment part 20 is inserted. According to an embodiment, the fastening nut 25, which has a box shape with four corners, may be seated within the main body housing 100, and be arranged to engage with the gear of the needle insertion depth adjustment part 20. According to an embodiment, the driving force of a first driving motor 21 may cause the fastening nut 25 to move forward or backward along the rotational axis due to engagement between the fastening nut 25 and the gear 22. That is, the engagement structure between the fastening nut 25 and the gear 22 may serve as a power conversion part that converts rotational force into rectilinear movement force.

According to an embodiment, the needle insertion depth adjustment part 20 may include the first driving motor 21, the gear 22, and a seating structure. According to an embodiment, the first driving motor 21 may serve as a power source that provides rotational force and may include a motor shaft 210. According to an embodiment, the first driving motor 21 may be seated in the main body housing by means of the seating structure.

According to an embodiment, the seating structure may include first and second seating support parts 101a and 101b, and a seating cover 23. According to an embodiment, the first and second seating support parts 101a and 101b may be formed in upright shapes as molded structures formed within the main body housing 100. According to an embodiment, the seating cover 23 may be arranged to surround the first driving motor 21 and may serve as a fixing member that secures the first driving motor 21 within the main body housing 100 by using screws (not shown).

According to an embodiment, the gear 22 may be a power transmission member that is fixed to the motor shaft 210 of the first driving motor 21 and rotates in accordance with the rotation of the first driving motor 21. According to an embodiment, the gear 22 may be arranged in engagement with the gear hole 252 of the fastening nut 25, thereby transmitting the power of the first driving motor 21 to the fastening nut 25. According to an embodiment, the gear 22 may be arranged in engagement with the gear hole 252 of the fastening nut 25, allowing the power of the first driving motor 21 to be transmitted to the fastening nut 25. For example, when the first driving motor 21 rotates clockwise, the fastening nut 25 may move forward toward the needle cartridge 40, and when the first driving motor 21 rotates counterclockwise, the fastening nut 25 may move backward toward the first driving motor 21.

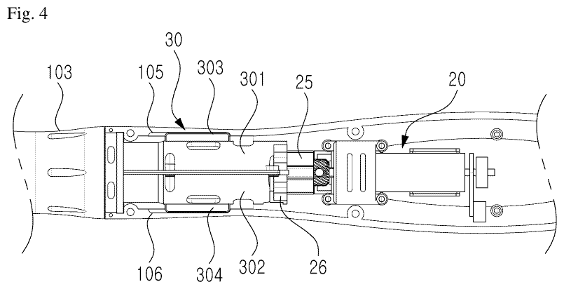

FIG. 4 is a plan view showing the assembled state of the needle insertion depth adjustment part of the handpiece for skin treatment according to an embodiment.

Referring to FIG. 4, according to an embodiment, the needle driving part 30 may be connected to one end of the needle insertion depth adjustment part 20 by the fastening nut 25 and the fastening member 26. According to an embodiment, the needle driving part 30 may be arranged between the needle cartridge 40 and the needle insertion depth adjustment part 20 within the main body housing 100, and may transmit a vibration force to the needle cartridge 40 so that the needle cartridge 40 reciprocates back and forth.

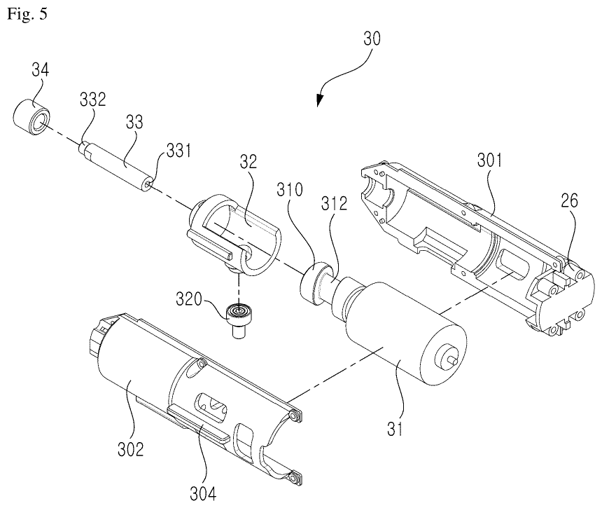

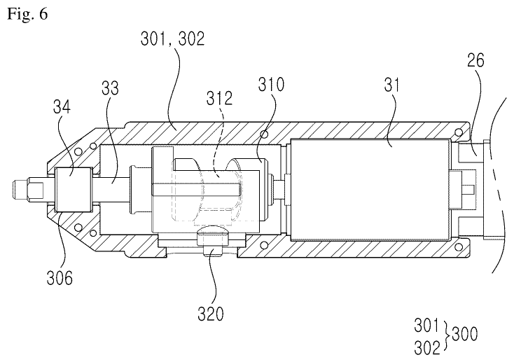

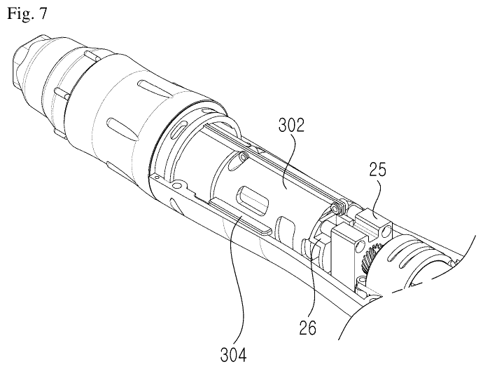

FIG. 5 is an exploded perspective view showing the configuration of a needle driving part of the handpiece for skin treatment according to an embodiment. FIG. 6 is a plan view showing the assembled configuration of the needle driving part of the handpiece for skin treatment according to an embodiment. FIG. 7 is a perspective view showing the assembled state of the needle driving part of the handpiece for skin treatment according to an embodiment.

Referring to FIGS. 5 to 7, according to an embodiment, the needle driving part 30 may serve as a driving source that provides the vibration force of the needles, and may be integrally fastened to the fastening nut 25 by the fastening member 26. According to an embodiment, the needle driving part 30 may include first and second motor covers 301 and 302. According to an embodiment, the first and second motor covers 301 and 302 may serve as a housing that accommodates a second driving motor 31. According to an embodiment, the first and second motor covers 301 and 302 may be seated by a guide structure within the housing (e.g., the housing 100 shown in FIG. 2) so as to be movable back and forth along the rotational axis.

According to an embodiment, the needle driving part 30 may include a cam unit configured to convert rotational force into rectilinear movement force. According to an embodiment, the cam unit may include a driving cam 310 and a driven cam 32. According to an embodiment, the driving cam 310 may be a cam member that rotates together with the second driving motor 31 while the second driving motor 31 is coupled to the motor shaft 210. According to an embodiment, the driven cam 32 may be a cam member that engages with the driving cam 310 and performs rectilinear movement according to the rotation of the driving cam 310.

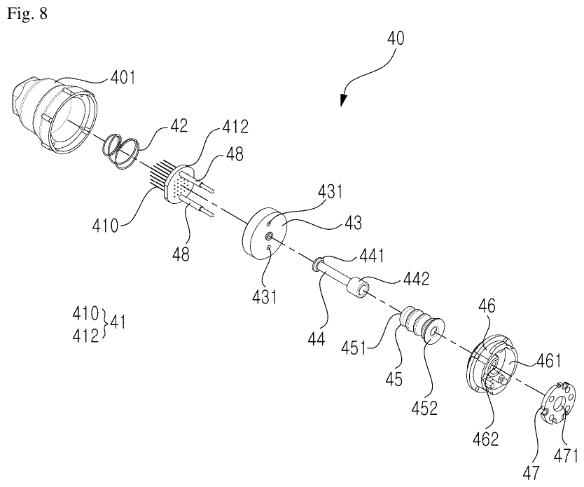

According to an embodiment, the driving cam 310 may have a helical cam groove 312 formed therein, and the driven cam 32 may include a cam protrusion 320 that engages with the cam groove 312. According to an embodiment, as the second driving motor 31 operates, the driving cam 310 may rotate clockwise, and simultaneously, the driven cam 32 may perform forward and backward movements. According to an embodiment, according to the operation of the driven cam 32, a vibration force that causes a needle tip of the needle cartridge 40 (e.g., the needle tip 41 shown in FIG. 8) to repeatedly move forward and backward may be transmitted thereto.

According to an embodiment, the needle driving part 30 may include a connection shaft 33 for transmitting the power of the driven cam 32 to the needle tip 41 (e.g., the needle tip 41 shown in FIG. 8). According to an embodiment, the connection shaft 33, which has a rod shape, has a first end 331 thereof fixed to the driven cam 32 and a second end 332 coupled to a piston shaft 44 of the needle cartridge 40 (e.g., the piston shaft 44 shown in FIG. 8). According to an embodiment, a portion of the connection shaft may be supported by a support bushing 34 that supports rectilinear movement. According to an embodiment, the support bushing 34, which has a ring-shaped cross-section, may have the form of a bushing, and be seated in a cylindrical seating recess 306 provided by the first and second motor covers 301 and 302. According to an embodiment, in the connection structure between the driven cam 32 and the connection shaft 33, the driven cam 32 and the connection shaft 33 are arranged concentrically (on a rotating axis) by the support bushing 34, thereby minimizing shaking during the operation of the second driving motor 31. For example, it may be preferable for the support bushing 34 to be arranged near the second end of the connection shaft 33.

According to an embodiment, the motor cover 300 includes the first motor cover 301 and the second motor cover 302, and the first and second motor covers may respectively include first and second guide protrusions 303 and 304 for guiding rectilinear movement caused by the operation of the needle insertion depth adjustment part 20. According to an embodiment, the first and second guide protrusions 303 and 304 protrude respectively in directions away from each other and may have a rectilinear shape extending in the direction of rectilinear movement (e.g., a forward and backward vibration direction).

According to an embodiment, first and second guide grooves 105 and 106 (e.g., the guide grooves 105 and 106 shown in FIG. 4) are formed in the first and second housings 101 and 102, respectively. The first and second guide protrusions 303 and 304 are inserted into the guide grooves 105 and 106, respectively, so that the guide protrusions 303 and 304 can perform limited rectilinear movement within the guide grooves 105 and 106.

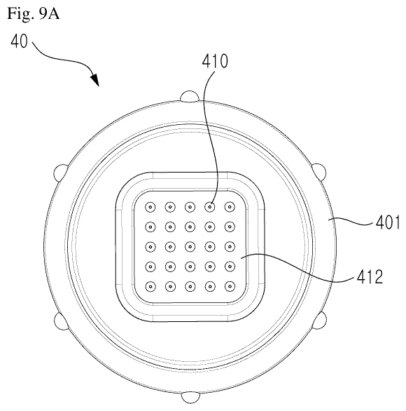

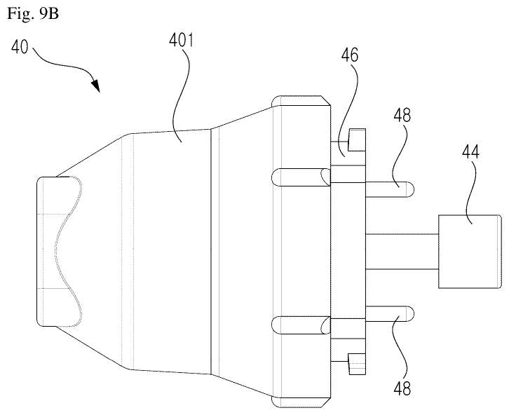

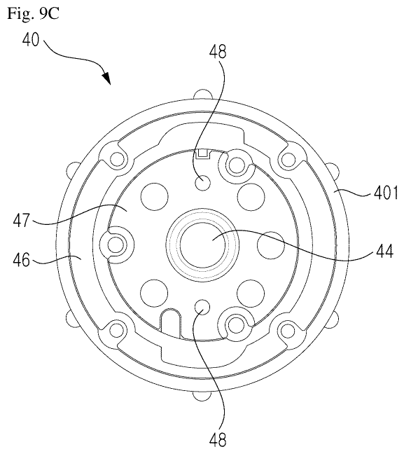

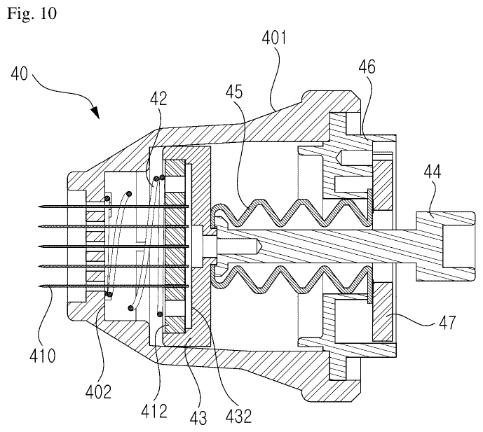

FIG. 8 is an exploded perspective view showing the configuration of a needle cartridge of the handpiece for skin treatment according to an embodiment. FIG. 9A is a front view showing the configuration of the needle cartridge of the handpiece for skin treatment according to an embodiment. FIG. 9B is a side view showing the configuration of the needle cartridge of the handpiece for skin treatment according to an embodiment. FIG. 9C is a rear view showing the configuration of the needle cartridge of the handpiece for skin treatment according to an embodiment. FIG. 10 is a front cross-sectional view showing the configuration of the needle cartridge of the handpiece for skin treatment according to an embodiment.

Referring to FIGS. 8 to 10, according to an embodiment, the needle cartridge 40 as a disposable and replaceable assembly may include the needle tip 41, the needle housing 401, an elastic body 42, a needle PCB 412, a piston housing 43, the piston shaft 44, a waterproof member 45, a cap 46, and a connection PCB 47. Among the components listed above, the needle tip 41 may be replaced after a single use.

According to an embodiment, the parts listed above may be classified as parts fixed to the main body housing 100 and vibrating parts that are not fixed to the main body housing 100 and move forward and backward. According to an embodiment, the parts fixed to the third housing 103 of the main body housing 100 may include the needle housing 401, the cap 46, and the connection PCB 47. According to an embodiment, the vibrating parts may be parts that are operatively coupled to the needle driving part 30 and perform the reciprocating movement of the needle driving part 30, and may include the needle tip 41, the piston housing 43, and the piston shaft 44.

According to an embodiment, the needle housing 401 may be coupled to the third housing 103 to protect the vibrating needle tip 41. According to an embodiment, the needle tip 41 may include a plurality of aligned needles 410 and the needle PCB 412. According to an embodiment, the plurality of needles 410 may be arranged upright at equal intervals on the first surface of the needle PCB 412. For example, sixteen needles 410 (e.g., the needles 410 shown in FIG. 9A) may be arranged at equal intervals on the first surface of the needle PCB.

According to an embodiment, the piston housing 43 may have a first surface coupled to the first end 441 of the piston shaft 44, and a second surface that serves as a seating member on which the needle tip 41 is seated. According to an embodiment, the piston housing 43 may have a coin-shaped seating recess 432 formed on the second surface thereof, wherein the coin-shaped needle PCB 412 may be seated in the seating recess 432. According to an embodiment, the cap 46 may be a part of a cover fastened to the second end of the needle housing 401, and may include a through hole 462 formed at a center thereof so that the piston shaft 44 passes therethrough, and a seating recess 461 in which the connection PCB 47 for high-frequency or low-frequency output is seated.

According to an embodiment, the waterproof member 45 may be a waterproof structure that seals the piston shaft 44 to prevent moisture ingress, and may be of a bellows type. According to an embodiment, the piston shaft 44 may pass through the waterproof member 45, and the waterproof member 45 may surround the piston shaft 44. According to an embodiment, the waterproof member 45 may be elastic and be compressed or extended along the direction of vibration. For example, the waterproof member may have an outwardly wrinkled shape with a plurality of corrugation portions, so that spacing between the corrugation portions decreases when the waterproof member is compressed and increases when the waterproof member is extended. For example, the waterproof member 45 may include a gasket. According to an embodiment, the first end of the waterproof member 45 may be arranged to be in close contact with the second surface of the piston housing 43, and the second end of the waterproof member 45 may be arranged to be in close contact with the first end of the connection PCB 47. According to an embodiment, the second end of the waterproof member 45 may be arranged to pass through the through hole of the cap 46.

According to an embodiment, the first end 441 of the piston shaft 44 may be fastened to the piston housing 43 by a fastener, and the second end 442 of the piston shaft 44 may be fixed to the connection shaft 33. According to an embodiment, the piston shaft 44 may be arranged to pass through the waterproof member 45, the piston housing 43, and the connection PCB 47. According to an embodiment, the piston shaft 44 may directly transmit the power of the needle driving part 30 to the needle tip 41. According to an embodiment, the piston shaft 44 performs back-and-forth vibration motion within the waterproof member 45 and may perform limited vibration motion within the needle housing 401. According to an embodiment, the piston shaft 44 may be sealed by the waterproof member 45 to minimize the generation of particles caused by the vibration of the piston shaft 44 generated during the vibration.

According to an embodiment, the connection PCB 47, which has an approximately coin-shaped form, may be seated in the seating recess 461 of the cap 46, and include the through hole 462 through which the second end 442 of the piston shaft 44 passes. According to an embodiment, the connection PCB 47 may be electrically connected to the needle PCB 412 via an electrical connection member, such as a conductive spring 48. According to an embodiment, the connection PCB 47 may be a PCB on which a component for low-frequency or high-frequency output is arranged and may output high-frequency or low-frequency to the needles 410 of the needle PCB 412 via the conductive spring 48.

According to an embodiment, the needle PCB 412 may have a plurality of upright needles 410 arranged by protruding on the first surface thereof and serve as a PCB for applying a positive or negative voltage to the needles. According to an embodiment, a voltage applied to the needles 410 generates pulses applied for the purpose of reversible electroporation, and the pulses may be unipolar, bipolar, or exponential, or may take the form of square waves or other shapes. Such pulses are determined according to the properties of the cell tissue, as well as the size and location of the selected cell tissue, so as to induce electroporation at specific sites within the body tissue, and may include pulses with uniform and sufficient voltage, current, density, duration, and frequency.

According to an embodiment, power supplied to the needles 410 may be an electric current having waveforms such as an alternating current (AC), a direct current (DC), or sinusoidal and cosinusoidal waves with magnitudes. The frequency may also be variously applied, including low, medium, or high frequencies, and stimulation pulses may be varied in multiple ways.

According to an embodiment, the elastic body 42 may be disposed between a step 402 formed at the entrance (an open portion) of the needle housing 401 and the needle PCB 412. According to an embodiment, the plurality of needles may be arranged to pass through the elastic body 42. For example, the elastic body 42 may be a compression coil spring with differing diameters at opposite ends thereof. According to an embodiment, the elastic body 42 may be arranged such that the first end of the elastic body 42 is in close contact with the step 402, and the second end thereof is in close contact with the needle PCB 412, so that the elastic body 42 may provide a force by which the elastic body 42 pushes the needle PCB 412 toward the cap 46.

According to an embodiment, one or more conductive springs 48 may be used to electrically connect the needle PCB 412 to the connection PCB 47. For example, the conductive spring 48 may be made of a metal material, and two conductive springs may be used. According to an embodiment, the conductive spring 48 may serve as both a conductor and a spring. According to an embodiment, the first end of the conductive spring 48 may be in contact with the second surface of the needle PCB 412, and the second end of the conductive spring 48 may be in contact with the first end of the connection PCB 47. According to an embodiment, in order to arrange the conductive springs 48, the piston housing 43 may include two through-holes 431, and the cap 46 may include two through-holes (not shown).

Referring to the drawings, in the operation of the handpiece 10 for skin treatment having the above-described structure, when the device is turned on, the driving cam 310 is rotated by the operation of the second driving motor 31. Simultaneously, the driven cam 32 generates vibration through back-and-forth rectilinear movement, and this vibrational force may be transmitted to the needle PCB 412 by the connection shaft 33 and the piston shaft 44.

Meanwhile, when it is necessary to adjust the insertion depth of the needles, turning on the first driving motor 21 causes the gear 22 to rotate. As the gear 22 rotates, the fastening nut 25, which is engaged with the gear 22, moves rectilinearly back and forth. This rectilinear movement causes the needle driving part 30, which is connected to the fastening member 26, to move rectilinearly back and forth. Consequently, the needle PCB 412 may also be capable of performing rectilinear back-and-forth movement. For example, the clockwise rotation of the first driving motor 21 may cause the needles 410 to move forward, while the counterclockwise rotation of the first driving motor may cause the needles 410 to move backward.

According to an embodiment, during the forward and backward movement of the piston housing 43, when the piston housing 43 advances toward the skin, air in the front portion (the space where the needles contacting the skin are located) is compressed, pushing against the skin. Subsequently, due to the positive pressure (compressed air) in the front portion, the needles 410 (which emit a high-frequency current) and the drug applied onto the skin are capable of simultaneously penetrating into the skin cells.

According to an embodiment, when the piston housing 43 is moved backward in the direction opposite to the skin, a slight negative pressure may be generated in the air inside the front portion.

According to an embodiment, the handpiece for skin treatment includes: the main body housing (e.g., the main body housing 100 shown in FIG. 2); the needle cartridge (e.g., the needle cartridge 40 shown in FIG. 8) including the needle tip which is accommodated by the needle housing (e.g., the needle housing 401 shown in FIG. 1) and includes the needle PCB (e.g., the needle PCB 412 shown in FIG. 8), on which the plurality of needles (e.g., the needles 410 shown in FIG. 8) are arranged in a protruding manner, with the needle cartridge 40 replaceably disposed on a first end of the main body housing; the needle driving part (e.g., the needle driving part 30 shown in FIG. 5) coaxially connected to the needle cartridge within the main body housing and configured to provide a vibration force to the needles; the needle insertion depth adjustment part (e.g., the needle insertion depth adjustment part 20 shown in FIG. 3) fastened coaxially to the needle driving part within the main body housing and configured to move the needle cartridge forward or backward; and at least one fastening member (e.g., the fastening member 25 and 26 shown in FIG. 3) integrally connecting the needle driving part and the needle insertion depth adjustment part to each other, wherein the needle insertion depth adjustment part may include the first driving motor (e.g., the first driving motor 21 shown in FIG. 3) seated in the main body housing; the gear (e.g., the gear 22 shown in FIG. 3) disposed on the shaft of the first driving motor (e.g., the motor shaft 210 shown in FIG. 3); and the fastening nut (e.g., the fastening nut 25 shown in FIG. 3) seated in the main body housing, engaged with the gear, and configured to move rectilinearly forward toward the needle cartridge and backward therefrom according to the operation of the first driving motor.

According to an embodiment, the needle cartridge (e.g., the needle cartridge 40 shown in FIG. 8) may include: the piston housing (e.g., the piston housing 43 shown in FIG. 8) disposed within the first side of the needle housing, with the needle PCB seated on the piston housing; the piston shaft (e.g., the piston shaft 44 shown in FIG. 8) having a first end coupled to the piston housing and a second end connected to the needle driving part; the cap (e.g., the cap 46 shown in FIG. 8) through which the piston shaft passes, with the cap coupled to the second side of the needle housing; the connection PCB (e.g., the connection PCB 47 shown in FIG. 8) seated on one surface of the cap and configured to generate ultrasonic waves; and at least one conductive spring (e.g., the conductive spring 48 shown in FIG. 8) electrically connecting the needle PCB and the connection PCB to each other.

According to an embodiment, the piston housing (e.g., the piston housing 43 shown in FIG. 8) may include at least one through-hole (e.g., the through-holes 431 shown in FIG. 8) through which the conductive spring passes.

According to an embodiment, the handpiece for skin treatment may further include the waterproof member (e.g., the waterproof member 45 shown in FIG. 8) arranged to surround the piston shaft (e.g., the piston shaft 44 shown in FIG. 8).

According to an embodiment, the waterproof member (e.g., the waterproof member 45 shown in FIG. 8) may be arranged such that the first end of the waterproof member is in close contact with one surface of the piston housing, and the second end thereof is in close contact with one surface of the connection PCB.

According to an embodiment, the waterproof member (e.g., the waterproof member 45 shown in FIG. 8) may include the plurality of corrugation portions, wherein the spacing between the corrugation portions may increase when the waterproof member is extended and decrease when the waterproof member is compressed.

According to an embodiment, the handpiece for skin treatment may further include the elastic body arranged such that the first end of the elastic body 42 is in close contact with the step formed in the needle housing (e.g., the needle housing 401 shown in FIG. 10), and the second end of the elastic body 42 is in close contact with the first surface of the needle PCB (e.g., the needle PCB 412 shown in FIG. 10).

According to an embodiment, the needle driving part (e.g., the needle driving part 30 shown in FIG. 5) may include: the motor cover (e.g., the motor cover 300 shown in FIG. 6) having the cylindrical seating recess (e.g., the seating recess 306 shown in FIG. 6) formed at one end portion thereof; the second driving motor (e.g., the second driving motor 31 shown in FIG. 5) seated in the motor cover; the driving cam (e.g., the driving cam 310 shown in FIG. 5) disposed on the shaft of the second driving motor; the driven cam (e.g., the driven cam 32 shown in FIG. 5) movably disposed within the motor cover, engaged with the driving cam, and configured to move rectilinearly within the motor cover according to the rotation of the driving cam; the connection shaft (e.g., the connection shaft 33 shown in FIG. 5) having a first end coupled to the driven cam and a second end coupled to the piston shaft, and configured to transmit a vibration force of the driven cam to the piston shaft; the support bushing (e.g., the support bushing 34 shown in FIG. 5) seated in the seating recess, coaxially arranged to surround the connection shaft, and configured to support the connection shaft; and the at least one guide protrusion (e.g., the guide protrusion 304 shown in FIG. 5) configured to guide the rectilinear movement of the driven cam.

According to an embodiment, the support bushing (e.g., the support bushing 34 shown in FIG. 5) has a ring-shaped cross-section, allows the connection shaft (e.g., the connection shaft 33 shown in FIG. 5) to pass therethrough, and is disposed in the seating recess to support the movement of the connection shaft.

The specific implementations described in the present disclosure are merely exemplary embodiments and are not intended to limit the scope of the disclosure in any way. For the sake of brevity, descriptions of conventional electronic components, control systems, software, and other functional aspects of such systems may be omitted.

In addition, in the present disclosure, the phrase “includes at least one of a, b, or c” may mean including only a, only b, only c, or any combination of two or more of a, b, and c (i.e., including a and b, b and c, a and c, or a, b, and c together).

Meanwhile, although the detailed description of the present disclosure has described specific embodiments, it is obvious that various modifications are possible within the scope of the present disclosure. Therefore, the scope of the present disclosure should not be limited to the described embodiments, but should be determined not only by the scope of the claims described below, but also by equivalents of the scope of the claims.

What is claimed is:

1. A handpiece for skin treatment, the handpiece comprising:
a main body housing;
a needle cartridge including the needle tip accommodated by a needle housing and having a needle PCB, on which a plurality of needles are arranged in a protruding manner, with the needle cartridge replaceably disposed on a first end of the main body housing;
a needle driving part coaxially connected to the needle cartridge within the main body housing and configured to provide a vibration force to the needles;
a needle insertion depth adjustment part fastened coaxially to the needle driving part within the main body housing and configured to move the needle cartridge forward or backward; and
at least one fastening member integrally connecting the needle driving part and the needle insertion depth adjustment part to each other,
wherein the needle insertion depth adjustment part comprises:
a first driving motor seated in the main body housing;
a gear disposed on a shaft of the first driving motor; and
a fastening nut seated in the main body housing, engaged with the gear, and configured to move rectilinearly forward toward the needle cartridge and backward therefrom according to an operation of the first driving motor.

2. The handpiece of claim 1, wherein the needle cartridge comprises:
a piston housing disposed within a first side of the needle housing, with the needle PCB seated on the piston housing;
a piston shaft having a first end coupled to the piston housing and a second end connected to the needle driving part;
a cap through which the piston shaft passes, with the cap coupled to a second side of the needle housing;
a connection PCB seated on one surface of the cap and configured to generate ultrasonic waves; and
at least one conductive spring electrically connecting the needle PCB and the connection PCB to each other.

3. The handpiece of claim 1, wherein a piston housing comprises at least one through-hole through which a conductive spring passes.

4. The handpiece of claim 2, further comprising:
a waterproof member arranged to surround the piston shaft.

5. The handpiece of claim 4, wherein the waterproof member is arranged such that a first end of the waterproof member is in close contact with one surface of the piston housing, and a second end thereof is in close contact with one surface of the connection PCB.

6. The handpiece of claim 4, wherein the waterproof member includes a plurality of corrugation portions, wherein spacing between the corrugation portions increases when the waterproof member is extended and decreases when the waterproof member is compressed.

7. The handpiece of claim 1, further comprising:
an elastic body arranged such that a first end of the elastic body is in close contact with a step formed in the needle housing and a second end of the elastic body is in close contact with one surface of the needle PCB.

8. The handpiece of claim 1, wherein the needle driving part comprises:
a motor cover having a cylindrical seating recess formed on one end portion thereof;
a second driving motor seated in the motor cover;
a driving cam disposed on a shaft of the second driving motor;
a driven cam movably disposed within the motor cover, engaged with the driving cam, and configured to move rectilinearly within the motor cover according to a rotation of the driving cam;
a connection shaft having a first end coupled to the driven cam and a second end coupled to a piston shaft, and configured to transmit a vibration force of the driven cam to the piston shaft;
a support bushing seated in the seating recess, coaxially arranged to surround the connection shaft, and configured to support a movement of the connection shaft; and
at least one guide protrusion configured to guide the rectilinear movement of the driven cam.

9. The handpiece of claim 8, wherein the support bushing has a ring-shaped cross-section, allows the connection shaft to pass therethrough, and is disposed in the seating recess to support the movement of the connection shaft.
